(19) United States
(12) Patent Application Publication
(10) Pub. No.: US 2011/0192401 A1
(43) Pub. Date: Aug. 11, 2011

(54) CLOSED RESPIRATORY SUCTION SYSTEM

(75) Inventors: Per Otto Børresen Gravesen, Tikob (DK); Jacob Ilskov, Valby (DK)

(73) Assignee: UNOMEDICAL A/S, Birkerod (DK)

(21) Appl. No.: 12/998,279

(22) PCT Filed: Nov. 4, 2009
(86) PCT No.: PCT/EP2009/064617

§ 371 Date: Apr. 5, 2011

(30) Foreign Application Priority Data
Nov. 4, 2008 (EP) 08168237.9

(51) Int. Cl. A61M 16/00; A62B 9/02

(52) U.S. Cl. 128/205.19; 128/205.24

(57) ABSTRACT

The present invention concerns a respiratory suction system for providing ventilation of a patient's respiratory tract, the system comprising an elongated catheter with a distal end, a manifold defining a flow path in a ventilator circuit and comprising access structure allowing the catheter to be advanced through the manifold and into a respiratory tract of a patient, the access structure including a valve which is openable in response to the advancement of the catheter through the valve and into the flow path. The valve has a proximal end has an annular outer flange section, a distal end and a tubular body extending between the proximal end and the distal end, the tubular body having a plurality of valve members extending from the annular flange section to the distal end which has end surfaces wherein a plurality of radial slits are provided, extending from the center towards the periphery and thereby separating the valve members at the distal end.

### FIELD OF THE INVENTION

The present invention relates to a closed respiratory suction system for providing ventilation of a patient's respiratory tract, the system comprising an elongated catheter with a distal end, a manifold defining a flow path in a ventilator circuit, and comprising access means allowing the catheter to be advanced through the manifold and into a respiratory tract of a patient, the access means comprising a valve which is openable in response to the advancement of the catheter through the valve and into the flow path.

### BACKGROUND ART

A respiratory suction system is known from EP 1 239 907 in which a valve is disposed within the manifold to selectively isolate the catheter from the ventilator circuit. The valve is moveable between an open position, wherein the valve permits the advancement of the catheter through the manifold, and a closed position, wherein the valve selectively isolates the catheter from the ventilator circuit. The valve is a hinged flap which is closing responsive to suction applied through the catheter when the catheter is not advanced through the manifold.

Examples of valves of the duckbill type are also known in relation to medical access devices from e.g. U.S. Pat. No. 5,456,284 and U.S. Pat. No. 6,439,541.

### SUMMARY OF THE INVENTION

An object of the present invention is to provide a respiratory system with a duckbill type valve with improved performance compared to known valves in such systems.

The above objects, together with numerous other objects, advantages, and features, which will become evident from the below description, are accomplished by a solution in accordance with the present invention by a closed respiratory suction system for providing suction of a patient's respiratory tract, the system comprising:
an elongated catheter with a distal end, and
a manifold defining a flow path in a ventilator circuit and comprising access means allowing the catheter to be advanced through the manifold and into a respiratory tract of a patient,
   the access means comprising a valve which is openable in response to the advancement of the catheter through the valve and into the flow path;
   the valve comprising:
a distal end,
a proximal end having an annular outer flange section, and
a tubular body having an inner side and an outer side and extending between the proximal end and the distal end, the distal end having an end portion, wherein a plurality of radial slits are provided, extending from the centre of the tubular body to a peripheral region, the slits having a closed rest position and being openable in response to the distal end of the catheter when the catheter is advanced towards the end portion of the valve in a direction from the proximal end of the valve.

According to the invention, a system is provided where the opening and the closure of the valve in response to the advancement and retraction, respectively, of the catheter are more precise, and the valve ensures central guidance of the catheter into the manifold. Moreover, the function of closure of the valve is achieved irrespective of any suction applied to the catheter.

In one embodiment, the plurality of radial slits may have an angle in relation to each other of less than 180° when viewed from one end of the valve.

In another embodiment, the plurality of radial slits may define one opening.

In yet another embodiment, the valve may have at least two slits having an angle in relation to each other of less than 180° when viewed from one end of the valve.

Furthermore, the tubular body may be adapted to open by opening the slits so that each side of the slit is separated from the other side of the slit at peripheral folds associated with each slit. The slits open in response to the advancement of the catheter in that the lips of the slit separate and the valve parts bend away from each other at the central end of the slits.

In addition, the valve may be formed so that the tubular body comprises valve sections extending from the slits towards the annular outer flange section.

Also, the valve sections may be equal in size and shape.

The tubular body may have an inner and an outer side, and each valve section preferably may have one or more tapered distal surfaces.

In a preferred embodiment, each valve section is provided with a protrusion on the inner side near the distal end of each valve section.

According to the invention, the protrusion may be a semi-spherical extension.

The protrusions may be abutting the distal end of the catheter, and due to the advancement of the catheter, the protrusions are displaced radially whereby the valve is opened. This is advantageous since the contact between the catheter surface and the valve is kept to a minimal as only the protrusions come into contact with the catheter. This entails a relatively low friction and thereby little obstruction when advancing the catheter through the valve.

In one embodiment, each slit may have a centre, and the protrusion is arranged substantially outside the centre.

In another embodiment, the protrusion may have a round shape facing the catheter when advanced through the valve.

In addition, the protrusion may be flexible to be able to bend inwards to receive the catheter.

Furthermore, there may be three valve sections, and the radial slits between the valve sections may be angularly equally spaced apart. In this way, self-centring catheter guidance is also provided in the valve. The area of the valve sections between the radial slits is preferably the same to ensure proper guidance and centring of the catheter.

Accordingly, in another embodiment, the valve may be rotary-symmetric.

Also, the valve may be made of an elastomeric material retaining its original shape. As an example, the valve may be made of silicone. Other types of material may alternatively be used for the valve, e.g. thermoplastic elastomers (TPE) such as soft polyvinylchloride (PVC).

In addition, the invention relates to a valve for a respiratory suction system for providing suction of a patient's respiratory tract, the system comprising:
an elongated catheter with a distal end, and
a manifold defining a flow path in a ventilator circuit and comprising access means allowing the catheter to be advanced through the manifold and into a respiratory tract of a patient,
   wherein the valve is openable in response to the advancement of the catheter through the valve and into the flow path;
   the valve comprising:
a distal end,
a proximal end having an annular outer flange section, and
a tubular body having an inner side and an outer side and extending between the proximal end and a distal end, the distal end having an end portion, wherein a plurality of radial slits are provided, extending from the centre of the tubular body to a peripheral region, the slits having a closed rest position and being openable in response to the distal end of the catheter when the catheter is advanced towards the end portion of the valve in a direction from the proximal end of the valve, wherein the plurality of radial slits has an angle in relation to each other of less than 180° when viewed from one end of the valve.

Finally, the invention relates to a valve as described above.

### BRIEF DESCRIPTION OF THE DRAWINGS

In the following, the invention is described in more detail with reference to the accompanying drawings, in which:

FIG. 1 is a schematic side view of a closed suction catheter assembly according to the invention;

FIG. 2 is a sectional view of the manifold of the suction catheter assembly;

FIG. 3 is a front view of a duckbill valve according to the invention;

FIG. 4 is a side view of same;

FIG. 5 is an end view of the valve;

FIG. 6 is a sectional side view of the valve;

FIG. 7 is a perspective view of the valve according to a preferred embodiment of the invention, and

FIGS. 8 and 9 are front views of the valve in an open position and a closed position, respectively.

### DETAILED DESCRIPTION OF THE INVENTION

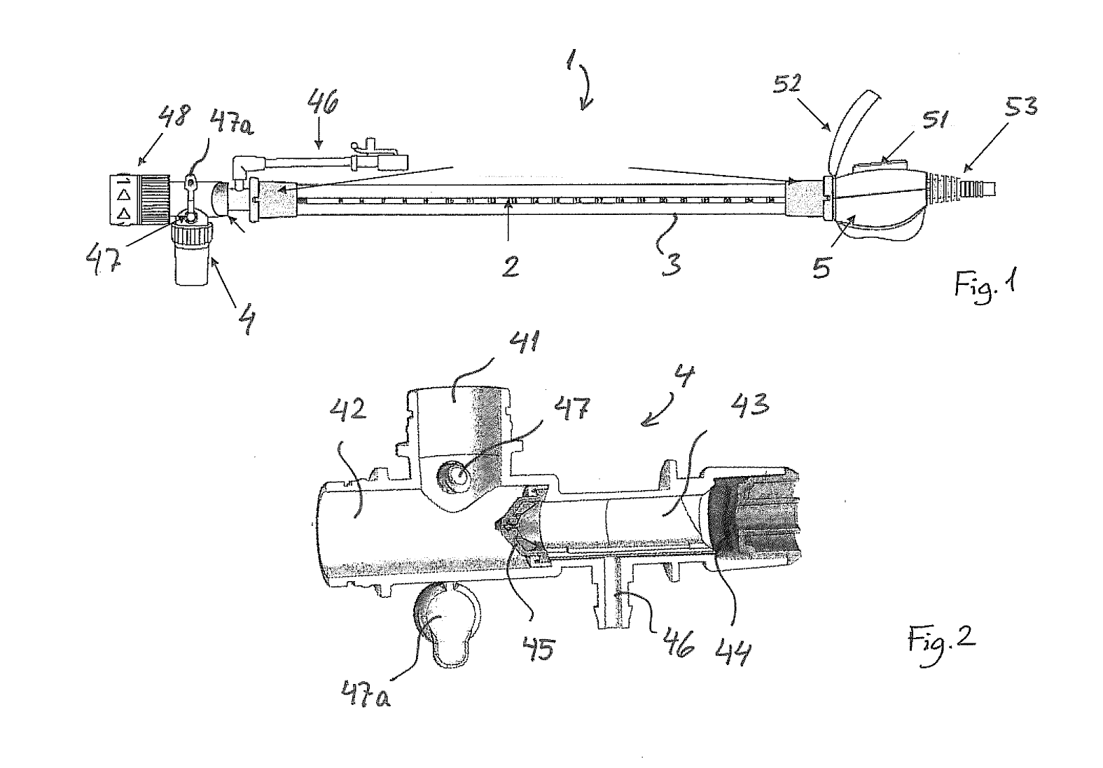

In FIG. 1, a closed suction catheter assembly 1 is shown. The assembly comprises a catheter 2 which is provided inside a protective, flexible sleeve 3 and is connected to a vacuum connecting member 5 at one end. At the other end, the catheter 2 is extendable into and through a patient ventilator manifold 4.

The vacuum connecting member 5 comprises a manually operated valve activated by a button 51, which, when pressed by an operator of the assembly, provides suction to the catheter 2 from a vacuum source (not shown) which is connected to a connection end 53. In order to avoid false activation of the suction, a protective cap 52 is provided to cover the activation button 51.

With reference to FIG. 2, the patient ventilator manifold 4 comprises a patient connector port 42 and a side port 41 suitable for connecting to a ventilation apparatus (not shown) in a conventional manner. The connector port 42 is provided with a suitable connection mechanism 48 for connecting the manifold 4 to ventilation tubes (not shown) provided in a patient. The ventilation tube may be a respiratory tube, also called an ET tube or an endotracheal tube. In the side port 41, an aerosol port 47 is arranged which is usable as a medication port with a cap 47a. Axially aligned with the connector port end 42 of the manifold 4 and opposite the side port 41, a catheter entry opening is provided. This opening includes a flushing chamber 43 and a duckbill type one-way valve 45 separating the respiratory flow path in the manifold, i.e. the flow path between ports 41 and 42, from the catheter 2 when retracted. At the opposite end of the valve 45, the flushing chamber 43 is preferably terminated by an annular wipe seal 44 sealing around the catheter 2 and wiping off any residuals on the catheter surface as the catheter 2 is retracted. The flushing chamber 43 is provided with a flushing port 46 to clean the catheter 2 and remove the wiped off residuals.

By the manifold 4, a flow path is defined in a ventilator circuit with access means allowing the catheter 2 to be advanced through the manifold 4 and into a respiratory tract of a patient. The valve 45 is openable in response to the advancement of the catheter 2 through the valve 45 and into the flow path and closable in response to the retraction of the catheter 2.

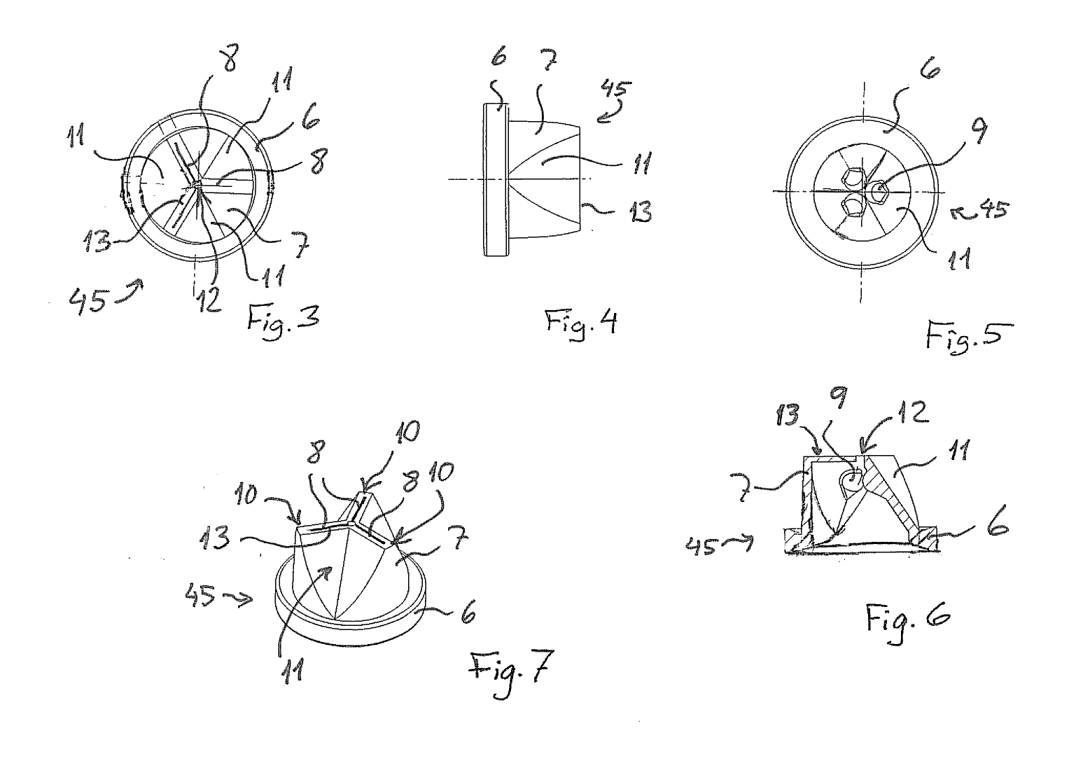

With reference to FIGS. 3 to 9, the valve 45 is of the duckbill type. The valve 45 is made of a resilient elastic material. The valve 45 is in its proximal end formed with an annular peripheral mounting flange 6 and a tubular body 7 extending from the annular outer flange section 6 towards the patient end of the closed suction assembly. The position of the valve 45 is shown in FIG. 2.

The valve may be made of an elastomeric material retaining its original shape. As an example, the valve may be made of silicone. Other types of material may alternatively be used for the valve, e.g. thermoplastic elastomers (TPE) such as soft polyvinylchloride (PVC).

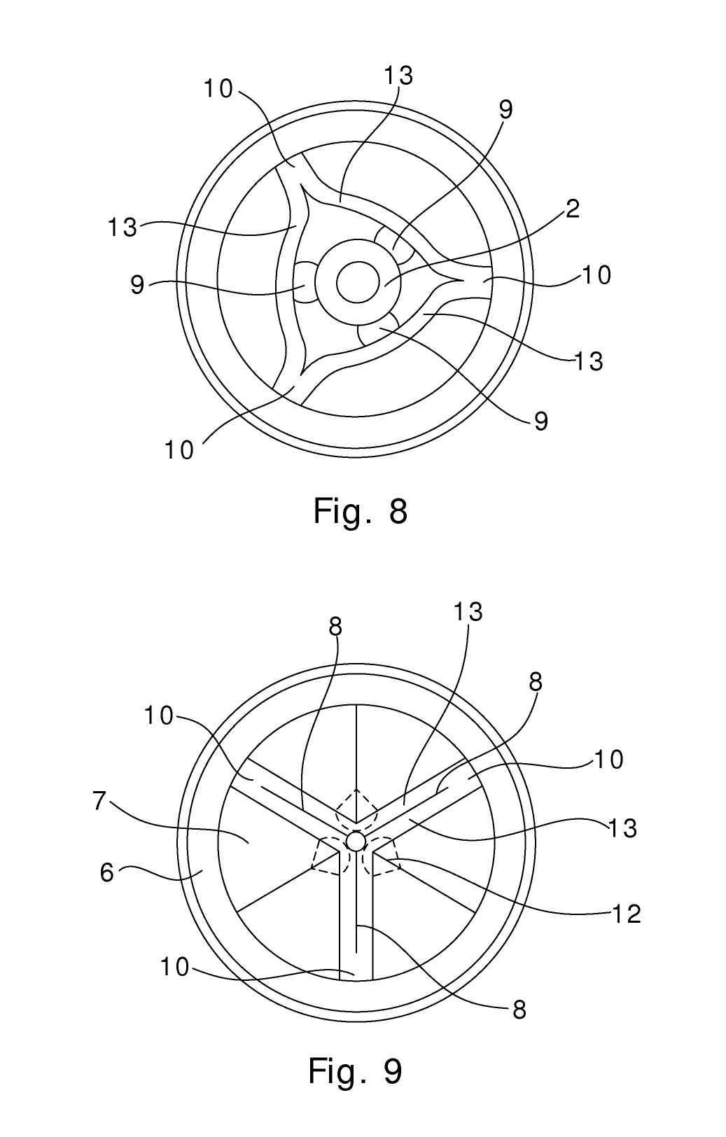

The tubular body 7 has an inner side and an outer side and is formed with a plurality of radial slits 8 in the end portion of the distal end 13 of the body 7. The slits extend from the centre of the end portion to the peripheral region of the tubular body. The slits have a closed position in which it rests before being forced open by a penetrating catheter. The body 7 is formed with valve sections 11 with tapered cavities 14 in the peripheral surface of the tubular body 7 and extends radially between the slits 8 in the end surface. The valve section cavities 14 are tapering from the mounting flange 6 towards the distal end 13. In the distal end 13, the slits 8 are provided radially so that at the extreme end, a fold-like peripheral slit end 10 is formed around which the slits 8 will open in response to the advancement of the catheter 2 (see FIG. 8).

On the inside of each valve section 11, a bulge or protrusion 9 is provided at the distal end inside the distal end 13 of the tubular body 7 (see in particular FIGS. 5 and 6). These protrusions 9 cooperate with the distal end of the catheter 2 and are pushed aside as the catheter 2 makes contact and is advanced through the valve (see FIGS. 8 and 9). The protrusion is arranged substantially outside at the middle of each slit and below the slit so as to support and centralise the catheter when advanced through the valve. As the resilient side walls of the cavities 14 in the tubular body 7 are pushed aside by the abutment of the catheter 2 against the protrusions 9, the slits 8 open as they fold open around the peripheral folds 10 on the distal end 13 (see FIGS. 8 and 9). In this way, the lips of each slit separate from each other as the slit opens.

The valve has three slits extending radially from the centre of the distal end of the valve towards the circumference of the tubular body. These slits define one opening extending radially outwards in three directions so that the angle between the directions is approximately 120°. The slits may thereby form an X-shape or a Y-shape.

In another embodiment, the plurality of radial slits has an angle in relation to each other of less than 180° when viewed from one end of the valve.

By having three slits, the catheter is supported by three points 15 when penetrating the valve, and the valve thereby provides centralising guidance of the catheter. Due to the fact that the sleeve 3 is flexible, it is important that the catheter is more firmly guided in the manifold so that the catheter enters the ventilation tube in the patient in a more controlled manner. Prior art duckbill valves only have one slit, and a catheter entering such a valve is not guided in a controlled manner, and the catheter is not centralised therein either.

The three protrusions 9 are situated on the inside of the valve 45, i.e. on the side of the valve 45 facing the flushing chamber 43. The protrusions 9 are preferably semi-spherically shaped and thereby ensure the opening and closing of the valve 45 in response to the catheter advancement or retraction, but without wiping residues on the catheter surface off the catheter on the duckbill valve 45 itself. These residues, secretions or mucous are removed by the flushing in the flushing chamber 43.

The semi-spherically shaped protrusion may be flexible so as to conform to the outside shape of the catheter 2 and thereby receive and control the catheter when it passes through the valve.

As shown in some of the figures, a small central hole 12 is created when the lips of the distal end 13 are closed, i.e. when the slits are closed. The small central hole 12 has a diameter of 0.05 mm to 0.08 mm. The three protrusions 9 are situated as close to the centre of the valve 45 as possible so that the suction catheter 2 extends there through, and the protrusions ensure the function of the catheter guidance and—by catheter retraction—leave residues on the catheter surface so that these are removed from the ventilator circuit 50 and brought into the flushing chamber 43. Before entering the sleeve, the catheter 2 is wiped free of secrete which is flushed away through the flushing port 46 and due to the protrusions not left in the ventilation circuit.

As shown in the figures, a preferred embodiment of the valve according to the invention comprises three angularly equally spaced slits 8, i.e. a valve with three valve sections, configured in angular symmetry (i.e. a rotary symmetric configuration). By the invention, it is realised that another number of radial slits and thereby another number of valve sections may be provided without departing from the scope of protection as defined in the claims.

1-15. (canceled)

16. A closed respiratory suction system for providing suction of a patient's respiratory tract, the system comprising:
an elongated catheter with a distal end, and
a manifold defining a flow path in a ventilator circuit and comprising access means allowing the catheter to be advanced through the manifold and into a respiratory tract of a patient,
the access means comprising a valve which is openable in response to the advancement of the catheter through the valve and into the flow path;
the valve comprising:
a distal end,
a proximal end having an annular outer flange section, and
a tubular body having an inner side and an outer side and extending between the proximal end and the distal end, the distal end having an end portion, wherein a plurality of radial slits are provided, extending from the centre of the tubular body to a peripheral region, the slits having a closed rest position and being openable in response to the distal end of the catheter when the catheter is advanced towards the end portion of the valve in a direction from the proximal end of the valve,
wherein the plurality of radial slits has an angle in relation to each other of less than 180° when viewed from one end of the valve.

17. A system according to claim 16, wherein the plurality of radial slits defines one opening.

18. A system according to claim 16, wherein the tubular body is adapted to open by opening the slits so that each side of the slit is separated from the other side of the slit at peripheral folds associated with each slit.

19. A system according to claim 16, wherein the tubular body comprises valve sections extending from the slits towards the annular outer flange section.

20. A system according to claim 19, wherein each valve section has one or more tapered distal surfaces.

21. A system according to claim 19, wherein each valve section is provided with a protrusion on the inner side of the tubular body near the distal end of each valve section.

22. A system according to claim 21, wherein each slit has a centre, and the protrusion is arranged substantially outside the centre.

23. A system according to claim 21, wherein the protrusion has a round shape facing the catheter when advanced through the valve.

24. A system according to claim 21, wherein the protrusion is flexible to be able to bend inwards to receive the catheter.

25. A system according to claim 19, wherein there are three valve sections.

26. A system according to claim 19, wherein the radial slits between the valve sections are angularly equally spaced apart.

27. A system according to claim 16, wherein the valve is made of an elastomeric material retaining its original shape.

28. A system according to claim 16, wherein the valve is made of silicone.

29. A valve for a respiratory suction system for providing suction of a patient's respiratory tract, the system comprising:
an elongated catheter with a distal end, and
a manifold defining a flow path in a ventilator circuit and comprising access means allowing the catheter to be advanced through the manifold and into a respiratory tract of a patient,
wherein the valve is openable in response to the advancement of the catheter through the valve and into the flow path;
the valve comprising:
a distal end,
a proximal end having an annular outer flange section, and
a tubular body having an inner side and an outer side and extending between the proximal end and a distal end, the distal end having an end portion, wherein a plurality of radial slits are provided, extending from the centre of the tubular body to a peripheral region, the slits having a closed rest position and being openable in response to the distal end of the catheter when the catheter is advanced towards the end portion of the valve in a direction from the proximal end of the valve,
wherein the plurality of radial slits has an angle in relation to each other of less than 180° when viewed from one end of the valve.

30. A system according to claim 29, wherein the plurality of radial slits defines one opening.

31. A system according to claim 29, wherein the tubular body is adapted to open by opening the slits so that each side of the slit is separated from the other side of the slit at peripheral folds associated with each slit.

32. A system according to claim 29, wherein the tubular body comprises valve sections extending from the slits towards the annular outer flange section.

33. A system according to claim 32, wherein each valve section has one or more tapered distal surfaces.

34. A system according to claim 32, wherein each valve section is provided with a protrusion on the inner side of the tubular body near the distal end of each valve section.

35. A system according to claim 34, wherein each slit has a centre, and the protrusion is arranged substantially outside the centre.

36. A system according to claim 34, wherein the protrusion has a round shape facing the catheter when advanced through the valve.

37. A system according to claim 34, wherein the protrusion is flexible to be able to bend inwards to receive the catheter.

38. A system according to claim 32, wherein there are three valve sections.

39. A system according to claim 32, wherein the radial slits between the valve sections are angularly equally spaced apart.

40. A system according to claim 29, wherein the valve is made of an elastomeric material retaining its original shape.

41. A system according to claim 29, wherein the valve is made of silicone.
